(19) United States
(12) Patent Application Publication
(10) Pub. No.: US 2026/0158044 A1
(43) Pub. Date: Jun. 11, 2026

(54) COMPOSITION FOR THE TREATMENT OF COVID-19

(71) Applicant: Albert Nguyen, Oklahoma City, OK (US)

(72) Inventor: Albert Nguyen, Oklahoma City, OK (US)

(21) Appl. No.: 19/183,354

(22) Filed: Apr. 18, 2025

Related U.S. Application Data
(62) Division of application No. 18/890,710, filed on Sep. 19, 2024.

(51) Int. Cl. A61K 31/573; A61K 9/00; A61K 47/10; A61K 47/26; A61K 47/38

(52) U.S. Cl. CPC A61K 31/573; A61K 9/0019; A61K 47/10; A61K 47/26; A61K 47/38

(57) ABSTRACT

A composition for the treatment of COVID-19 is disclosed, including a first composition including approximately 10 mg of dexamethasone. The first composition is combined with a second composition including between approximately 48 mg to approximately 80 mg of triamcinolone acetonide, sodium chloride, benzyl alcohol, carboxymethylcellulose sodium, and polysorbate 80.

### CROSS-REFERENCE TO RELATED APPLICATIONS

The present application claims priority to U.S. Nonprovisional application Ser. No. 18/890,710 filed Dec. 11, 2020, titled “COMPOSITION FOR THE TREATMENT OF COVID-19,” which is hereby incorporated by reference in its entirety.

### TECHNICAL FIELD

The embodiments disclosed herein generally relate to a composition for the treatment of COVID-19.

### BACKGROUND

Coronavirus SARS-CoV-2, the causative agent of Coronavirus disease 2019 (COVID-19) is a contagious virus known to infect humans. The symptoms of COVID-19 are highly variable and range from mild to severe, with the potential for causing death, especially in sensitive populations. In such, COVID-19 has had a significant impact on healthcare systems worldwide.

To mitigate risk, several vaccines have been developed and approved in various countries. Preventative measures for reducing the likelihood of exposure to the virus include physical and social distancing, quarantining, ventilation of indoor spaces, covering coughs and sneezes, hand washing, among others. While drugs have been developed, primary treatment is mainly symptomatic in nature. COVID-19 infection is known to cause high levels of inflammation in the human body.

Current anti-inflammatory treatment for COVID-19 patients include Dexamethasone at a dose of 6 mg administered daily for ten days. However, this treatment plan is reserved for patients having severe symptoms. Higher dexamethasone doses, as shown in clinical trial testing, resulted in an increase in deaths in the treatment group, especially in mild and moderate symptom patients. This is caused by the necessity to “wean down” from the high dose steroid to avoid adrenal crisis, hypotension, and/or death. Another study has revealed that Nirmatrelvir/ritonavir (sold under the brand name Paxlovid), is ineffective in relieving symptoms in patients having mild to moderate symptoms. In such, no treatment for COVID-19 infected patients is available for patients having mild or moderate symptoms.

The Center for Disease Control has established criteria which defines symptoms severity in COVID-19 infected patients. Mild symptoms included fever, cough, sore throat, headache, muscle pain, and gastrointestinal symptoms without dyspnea. Moderate symptoms included any symptoms of mild illness and dyspnea with exertion. In addition, patients who had a respiratory rate of breaths/minute, oxygen saturation of on room air, and pulse rate of beats/minute were categorized as having moderate symptoms. Severe symptoms include any symptoms of mild/moderate illness, dyspnea at rest, or respiratory distress. Clinical signs of severe illness included a respiratory rate of >30 breaths/min, oxygen saturation of <93% on room air, and pulse rate of 125 beats/min.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein relate to a composition for the treatment of COVID-19, including a first composition including approximately 10 mg of dexamethasone. The first composition is combined with a second composition including between approximately 48 mg to approximately 80 mg of triamcinolone acetonide, sodium chloride, benzyl alcohol, carboxymethylcellulose sodium, and polysorbate 80.

The combined composition includes two steroids to alleviate the need for weaning down following the administration of dexamethasone, thereby reducing the side effects caused by high doses of dexamethasone and creating a safe and effective treatment for COVID-19. The composition is especially effective in treating patients having mild or moderate symptoms associated with COVID-19 infection. The act of ‘wean-down’ the steroid at higher dosing inadvertently add more steroid to the human body and lead to side effect such as immunosuppression and the development of secondary pneumonia. The composition disclosed herein overcomes this limitation in the prior art.

In one aspect, the second composition further comprises sodium hydroxide.

In one aspect, the second composition further comprises hydrochloric acid.

In one aspect, the pH of the second composition is between approximately 5.0 and approximately 7.5.

A method for the treatment of COVID-19 is also disclosed using the composition. The method includes forming a first composition which includes approximately 10 mg of dexamethasone. A second composition is formed which includes approximately 48 mg to approximately 80 mg of each of the following: approximately 48 mg to approximately 80 mg of triamcinolone acetonide, sodium chloride, benzyl alcohol, carboxymethylcellulose sodium and polysorbate 80. The combined composition is drawn into a syringe and is injected into the patient.

Administration of the combined composition is administered once per day via injection using a syringe. Subsequent administration of the combined composition may continue each day.

For patients having mild symptoms, the combined composition is injected once every two days until symptomatic resolution. For patients having moderate symptoms, the combined composition is injected once every two days until symptomatic resolution. For patients having severe symptoms, the combined composition is injected once every day until symptomatic resolution.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a flowchart of a method for forming and administering a composition effective in the treatment of COVID-19, according to some embodiments.

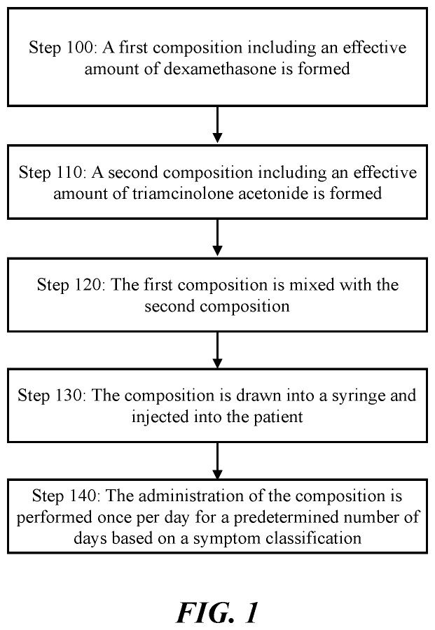

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a composition used in the treatment of COVID-19 infection. The composition includes a first composition comprising an effective amount of a first steroid. The first steroid is an effective amount of dexamethasone. The composition also includes a second composition including a second steroid. The second steroid is an effective amount of triamcinolone acetonide. The second composition also includes a sufficient amount of sodium chloride, a sufficient amount of benzyl alcohol, a sufficient amount of carboxymethylcellulose sodium, and a sufficient amount of polysorbate 80. The combination of dexamethasone and triamcinolone allows for a once-a-day injection without the need to “wean down” the steroid. This is possible because triamcinolone is a sustained-release steroid which can be detected in the human body up to three weeks following its intramuscular administration. The following criteria, as established by the Center for Disease Control, is used to define patients having mild and moderate symptoms. Mild symptoms included fever, cough, sore throat, headache, muscle pain, and gastrointestinal symptoms without dyspnea. Moderate symptoms included any symptoms of mild illness and dyspnea with exertion. In addition, patients who had a respiratory rate of breaths/minute, oxygen saturation of on room air, and pulse rate of beats/minute were categorized as having moderate symptoms. Severe symptoms included any symptoms of mild/moderate illness, dyspnea at rest, or respiratory distress. Clinical signs of severe illness included a respiratory rate of >30 breaths/min, oxygen saturation of <93% on room air, and pulse rate of 125 beats/min. One skilled in the arts will readily understand that the classification of symptoms (i.e., mild symptoms or moderate symptoms) may be adjusted based on the physician's interpretation of the presenting symptoms shown by the patient. In such, the above classifications may be modified.

The disclosed system relates to compositions and methods for the treatment of COVID-19 by using a combined intramuscular injection of dexamethasone and triamcinolone acetonide in specified quantities and ratios, along with additional excipients to stabilize the formulation. The composition is designed for administration through a single 3 mL syringe to provide symptom relief for patients diagnosed with COVID-19, particularly across a range of symptom severities classified as mild, moderate, or severe.

A first composition may be formed by measuring and isolating approximately 10 milligrams (mg) of dexamethasone. Dexamethasone is a corticosteroid commonly used for its anti-inflammatory and immunosuppressive effects. In this application, dexamethasone may serve as the fast-acting component of the therapeutic formulation, intended to rapidly reduce inflammation associated with SARS-CoV-2 infection. The first composition may be prepared in a sterile liquid form suitable for intramuscular administration and may be drawn from a vial or ampule using aseptic technique.

Separately, a second composition may be formulated to include triamcinolone acetonide in an amount ranging from approximately 48 mg to approximately 80 mg. Triamcinolone acetonide is a synthetic corticosteroid with a sustained-release profile when administered intramuscularly. It may function as a longer-acting steroidal component in the formulation, reducing the need for tapering or “weaning down” typically required when administering high doses of fast-acting steroids like dexamethasone alone. This characteristic may reduce the likelihood of adverse effects such as adrenal suppression or rebound inflammation.

In addition to triamcinolone acetonide, the second composition may include excipients that support isotonicity, sterility, and solubility. These excipients may include sodium chloride at approximately 0.66% w/v, which may provide isotonic conditions suitable for injection. Benzyl alcohol at approximately 0.99% w/v may act as a preservative to inhibit microbial growth. Carboxymethylcellulose sodium at approximately 0.63% may be included as a thickening or suspending agent, which may help maintain uniform dispersion of triamcinolone particles in the suspension. Polysorbate 80 at approximately 0.04% may function as a surfactant, enhancing the solubility and stability of the steroidal component.

In some embodiments, the second composition may further include pH-adjusting agents such as sodium hydroxide or hydrochloric acid. These agents may be used to bring the pH of the formulation within a physiologically compatible range of approximately 5.0 to 7.5. Maintaining the pH within this range may optimize steroid solubility and reduce the risk of injection site irritation.

The method of administration may begin by drawing the 10 mg dexamethasone first composition into a 3 mL syringe. Afterward, the second composition containing triamcinolone acetonide and the supporting excipients may also be drawn into the same syringe, thereby forming a mixed composition just prior to administration. This approach may simplify dosing and reduce the number of injections required for effective treatment.

Once the combined formulation is prepared in the syringe, it may be injected intramuscularly into the patient. The injection site may vary depending on practitioner discretion, with common locations including the deltoid or gluteal muscles. The composition may be administered once per day, and repeated injections may follow according to the severity of the patient's COVID-19 symptoms.

For patients classified as having mild symptoms, such as fever, cough, sore throat, and mild gastrointestinal symptoms without dyspnea, the therapeutic dose may consist of 10 mg dexamethasone combined with 48 mg of triamcinolone acetonide. This dosage may be administered once on the first day of treatment, and then once every two days until the patient's symptoms resolve.

In patients experiencing moderate symptoms, which may include dyspnea on exertion, elevated respiratory rate, or oxygen saturation between 93-95% on room air, the administered composition may contain 10 mg of dexamethasone and 60 mg of triamcinolone acetonide. Similar to the mild protocol, administration may occur once per day initially, followed by once every two days until clinical resolution of symptoms.

In cases where patients present with severe COVID-19 symptoms, such as dyspnea at rest, respiratory distress, or oxygen saturation levels below 93%, the combined dose may contain 10 mg dexamethasone and 80 mg triamcinolone acetonide. The dosage may be administered once daily and repeated every 24 hours until symptoms subside.

Triamcinolone acetonide's pharmacokinetics may allow for its persistence in the body for several days to weeks, thus supporting its role in mitigating the requirement for tapering doses as is typically done when high levels of dexamethasone are used. This extended duration of action may help manage inflammation over the course of the infection without subjecting patients to the cumulative steroid burden associated with prolonged high-dose corticosteroid use.

In clinical practice, physicians may adjust the triamcinolone dose within the stated range based on the patient's age, comorbidities, or other risk factors. While the 10 mg dose of dexamethasone remains constant across mild, moderate, and severe use cases, the flexible triamcinolone dosing protocol enables tailored treatment based on clinical assessment.

The combined use of dexamethasone and triamcinolone acetonide, delivered via intramuscular injection and supported by a stable delivery medium, may provide a composition that achieves both immediate symptom relief and prolonged anti-inflammatory control. The formulation avoids complications arising from abrupt withdrawal or prolonged use of a single high-potency steroid and may thus offer a simplified treatment pathway for practitioners managing COVID-19 cases in outpatient or resource-limited settings.

The described method and composition may be manufactured and stored using standard sterile technique in accordance with compounding pharmacy or Good Manufacturing Practice (GMP) protocols. The formulations described herein may be prepared extemporaneously or stored in ready-to-use injection kits, as long as stability, sterility, and regulatory requirements are maintained.

Although the compositions and methods described herein may be particularly applicable to COVID-19 treatment, one skilled in the art may recognize that similar formulations could be evaluated for other inflammatory or viral respiratory conditions, subject to appropriate testing and clinical evaluation.

The steroid injection provides symptomatic relief within two to four days in mild to moderate symptoms patients.

In some embodiments, the effective amount of dexamethasone is approximately 10 mg. In such, the effective amount of the first composition is 10 mg.

In some embodiments, the effective amount of triamcinolone acetonide is between approximately 40 mg to 80 mg.

In some embodiments, the sufficient amount of sodium chloride is approximately 0.66⁰ 0 for isotonicity.

In some embodiments, the sufficient amount of benzyl alcohol is approximately 0.99% (w/v) as a preservative.

In some embodiments, the sufficient amount of carboxymethylcellulose sodium is approximately 0.63%.

In some embodiments, the sufficient amount of polysorbate 80 is approximately 0.04%. In some embodiments, the sufficient amount of sodium hydroxide may be present to

adjust pH to 5.0 to 7.5.

In some embodiments, the sufficient amount of hydrochloric acid may be present to adjust pH to 5.0 to 7.5.

Sodium chloride, benzyl alcohol, carboxymethylcellulose sodium, and polysorbate 80 are included as a delivery mechanism for triamcinolone acetonide.

FIG. I illustrates a flowchart of a method for the treatment of COVID-19 using the composition. In step 100, a first composition which includes approximately 10 mg of dexamethasone. In step 110, a second composition is formed which includes approximately 48 mg to approximately 80 mg of each of the following: approximately 48 mg to approximately 80 mg of triamcinolone acetonide, sodium chloride, benzyl alcohol, carboxymethylcellulose sodium and polysorbate 80. In step 120, the first composition is mixed with the second composition. In step 130, the composition is drawn into a 3 mL syringe and is injected into the patient. In step 140, the composition is administered once per day for a continuous period of days, depending on the severity of the patients'symptoms.

Administration of the combined composition is administered once per day via injection using a syringe. Subsequent administration of the combined composition may continue each day.

For patients having mild symptoms, the combined composition is injected once per day every two days. For patients having moderate symptoms, the combined composition is injected once per day every two days.

In some embodiments, patients who have mild symptoms of COVID-19 and who receive the injection of the combined composition for a period of every two days may experience diminishing or complete resolution of symptoms after three to four days.

In some embodiments, patients having mild symptoms of COVID-19 infection may receive approximately 10 mg of dexamethasone combined with 48 mg of triamcinolone acetonide.

In some embodiments, patients having moderate symptoms of COVID-19 infection may receive approximately 10 mg of dexamethasone combined with 60 mg of triamcinolone acetonide.

In some embodiments, patients having severe symptoms of COVID-19 infection may receive approximately 10 mg of dexamethasone combined with 80 mg of triamcinolone acetonide.

In some embodiments, patients who have moderate symptoms of COVID-19 and who receive the injection of the combined composition for a period of every two days may experience diminishing or complete resolution of symptoms after six to eight days.

Effective amounts of each of the dexamethasone and triamcinolone acetonide may be determined by the physician based on the symptoms being presented by the patient. An effective amount of 10 mg of dexamethasone may be utilized for patients having mild, moderate, or severe symptoms. The effective amount of 48 mg of triamcinolone acetonide may be used for patients having mild symptoms. The effective amount of 60 mg of triamcinolone acetonide may be used for patients having moderate symptoms. The effective amount of 80 mg triamcinolone acetonide may be used for patients having severe symptoms.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations.

The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and/or describing embodiments of this disclosure. Modifications and adaptations to these embodiments will be apparent to those skilled in the art and may be made without departing from the scope or spirit of this disclosure.

As used herein and in the appended claims, the singular forms “a”, an′ and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “combine” “mix” (or any of their forms) are used interchangeably herein and, in both cases, are generic to the direct coupling of two entities (without any nonnegligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim

Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. A method for the treatment of COVID-19, the method comprising the steps of:
forming a first composition, comprising:
approximately 10 mg of dexamethasone; and forming a second composition comprising approximately 48 mg to approximately 80 mg of the following:
approximately 48 mg to approximately 80 mg of triamcinolone acetonide; sodium chloride; benzyl alcohol; carboxymethylcellulose sodium; and polysorbate 80; drawing the first composition into a 3 mL syringe;
drawing the second composition into the 3 mL syringe; and injecting the combined composition from the syringe into a patient.

2. The method of claim 1, wherein the second composition further comprises sodium hydroxide.

3. The method of claim 1, wherein the second composition further comprises hydrochloric acid.

4. The method of claim 1, wherein the pH of the second composition is between approximately 5.0 and approximately 7.5.

5. The method of claim 1, wherein an effective amount of the composition for the treatment of the patient having mild symptoms of COVID-19 infection comprises:
10 mg of dexamethasone; and
48 mg of triamcinolone,
wherein the effective amount of the combined composition is administered once per day; and once every subsequent two days until symptomatic resolution.

6. The method of claim 1, wherein an effective amount of the composition for the treatment of the patient having moderate symptoms of COVID-19 infection comprises:
10 mg of dexamethasone; and
60 mg of triamcinolone, wherein the effective amount of the combined composition is administered once per day; and once every subsequent two days until symptomatic resolution.

7. The method of claim 1, wherein an effective amount of the composition for the treatment of the patient having severe symptoms of COVID-19 infection comprises:
10 mg of dexamethasone; and
80 mg of triamcinolone, wherein the effective amount of the combined composition is administered once per day; and once every subsequent day until symptomatic resolution.

8. The method of claim 1, wherein the second compound is comprised of:
0.66% sodium chloride;
0.99% benzyl alcohol;
0.63% carboxymethylcellulose sodium; and
0.04% polysorbate 80.
